(19) United States
(12) Patent Application Publication
(10) Pub. No.: US 2008/0241121 A1
(43) Pub. Date: Oct. 2, 2008

(54) INHIBITORS OF THE CERAMIDE METABOLIC PATHWAY AS ADJUNCTS TO OPIATES FOR PAIN

(76) Inventor: Daniela Salvemini, Chesterfield, MO (US)

Correspondence Address: RANDOLPH BRETTON, 2440 PRO TOUR DR., BELLEVILLE, IL, 62220, US

(21) Appl. No.: 11/695,519

(22) Filed: Apr. 2, 2007

(51) Int. Cl. A61K 38/46

(52) U.S. Cl. 424/94.6

(57) ABSTRACT

A method for treating opiate induced tolerance or opiate induced hyperalgesia in a subject is closed. More specifically, the method provides for reducing ceramide levels with an agent thereby treating opiate induced antinociceptive/analgesic tolerance or hyperalgesia in a human or non-human subject. The method further allows for improved pain management in subjects suffering from chronic pain as well as treatment for opiate induced disorders.

### BACKGROUND

1. Field of the Invention

This invention relates generally to compositions and methods of treating opiate induced tolerance or opiate induced hyperalgesia in a subject. Specifically, the invention is directed to compositions and methods for treating opiate induced antinociceptive/analgesic tolerance or hyperalgesia by inhibiting ceramide synthesis or reducing ceramide levels in a subject.

2. Description of the Related Art

Prolonged use of opiates results in antinociceptive tolerance, such that higher doses are required to achieve equivalent analgesia (1) or antinociception (2-4). Adaptative modifications in cellular responsiveness and, particularly, desensitization and down-regulation of opioid receptors are at the origin of this phenomenon (5). By contrast, an alternative hypothesis is that stimulation of opioid receptors over time triggers activation of anti-opioid systems that, in turn, reduce sensory thresholds, thereby resulting in hypersensitivity to tactile stimulation (i.e. allodynia) and to noxious thermal stimulation (i.e hyperalgesia) (3, 6, 7). As a corollary to this hypothesis, such opioid induced hypersensitivity paradoxically diminishes the net analgesic effect of the opioid agonist (3, 6, 7). Support for this alternative hypothesis has been evidenced in vivo in animals (8, 9, 10) and in humans (11, 12, 13). Thus, it is thought that analgesic tolerance arises when pain facilitatory systems become sensitized or hyperactive after repeated opioid use. In other words hyperalgesia and antinociceptive/analgesic tolerance are a result of the same disorder stemming from opiate use. However, the exact mechanisms by which prolonged opiate exposure induces hyperalgesia and tolerance remain unclear.

Ceramide is a sphingolipid signaling molecule which is generated from de novo synthesis coordinated by serine palmitosyltransferase (SPT) and ceramide synthase (CerS) and, or by enzymatic hydrolysis of sphingomyelin by sphingomyelinases (SMases). The de novo pathway is stimulated by numerous chemotherapeutics and usually results in prolonged ceramide elevation. Ultimately, the steady-state availability of ceramide is regulated by ceramidases that convert ceramide to sphingosine by catalyzing hydrolysis of its amide group. One form of acid ceramidase may also be a secreted enzyme, while a form of neutral ceramidase may be mitochondrial and hence might affect ceramide synthase-mediated ceramide signaling in that compartment.

Ceramide is also generated by enzymatic hydrolysis of sphingomyelin by sphingomyelinases. Sphingomyelin is generated by the enzyme sphingomyelin synthase (SMS) and localizes to the outer leaflet of the plasma membrane, providing a semipermeable barrier to the extracellular environment (14). Several isoforms of sphingomyelinase can be distinguished by pH optima for their activity, and referred to as acid (ASMase), neutral (NSMase) or alkaline SMase. Of these isoforms, NSMase and ASMase, are rapidly activated by diverse stressors and cause increased ceramide levels within minutes to hours. Mammalian ASMase and NSMase have been cloned from distinct genes (15). ASMase, was originally described as a lysosomal enzyme (pH optimum 4.5-5) that is defective in patients with Niemann-Pick disease. More recently, a secretory isoform was also identified that targets the plasma membrane, and is secreted extracellularly (16, 17) (FIG. 1). Derived from the same inactive 75 kDa precursor, the lysosomal and secretory ASMase differ by their NH2-termini and display different glycosylation patterns, that likely determines their targeting. Secretory ASMase hydrolyzes cell surface sphingomyelin to initiate signaling (16, 17) whereas neutral SMase is primarily located to the plasma membrane. Consequently, each SMase generates separate intercellular pools of ceramide.

### SUMMARY

A method of treating opiate induced antinociceptive/analgesic tolerance or opiate induced hyperalgesia in a subject. The method generally includes reducing ceramide in the subject by administering an agent.

Other aspects and iterations of the invention will in part be apparent and in part pointed out hereinafter.

### BRIEF DESCRIPTION OF THE DRAWINGS

FIG. 1 depicts a schematic that illustrates the ceramide metabolic pathway. Chronic administration of morphine activates the ceramide metabolic pathway leading to increases in ceramide levels at various levels of the neuroaxis. This increase leads to the events culminating in the development of antinociceptive tolerance and hyperalgesia known to occur in man and animals after morphine for the management of chronic pain of various etiologies.

FIG. 2 depicts a graph that illustrates the inhibition of antinociceptive tolerance by inhibition of ceramide synthesis using Fumonisin B1 (FB1), tyclodecan-9-xanthogenate (D609), and myriocin in mice. When compared to the control groups, repeated administration of morphine, led to the development of antinociceptive tolerance as evidenced by a reduction in hot plate latency change to acute injection of morphine on day 5. FB1, myriocin (Myr) or D609 when given together with morphine attenuated tolerance. These agents when given alone did not potentiate the antinociceptive responses to the acute dose of morphine in control animals. *P<0.001 for Morphine groups vs Control groups; † P<0.001 for Morphine+drug treated groups vs Morphine groups.

FIG. 3 depicts a series of photomicrophgraphs that illustrate the reduction of ceramide in the spinal column of mice after treatment with morphine and ceramide synthesis inhibitor FB1. Immunohistochemical techniques were used for detection of ceramide. No positive staining for ceramide was observed in the dorsal horn when compared to ventral horn tissues of control groups (a, a1, a2). Five days after morphine treatment, a marked appearance of positive staining for ceramide (brown) was observed in the dorsal horn when compared to the ventral horn (b, b 1, b2 see arrows). FB1 treatment abolished the presence of positive staining for ceramide (c, c1, c2). Tissue sections were stained using 3,3′-diaminobenzidine (DAB). Representative of at least 3 experiments performed on different days. Tissues from the dorsal and ventral spinal cord were taken on the same day and processed together.

FIG. 4 depicts a graph illustrating that co administration of FB1 with morphine blocks an increase in ceramide levels that occurs with morphine alone. Repeated administration of morphine (Mor) increased the levels of 18:0, 20:0 and 22:0 ceramide as measured on day five using ESI-MS/MS. Co-administration with FB1 attenuated these changes. Results are expressed as % increase from control values (n=3). *P<0.05 when compared to morphine alone.

### DETAILED DESCRIPTION

The present invention provides a method of effective pain management for patients suffering from pain, including chronic pain of various etiologies without the unwanted side effects that are seen when opiates are administered alone. The present invention also provides a method of reducing the total amount of opiate necessary to relieve pain in a human or animal subject over the course of long-term opiate administration. The present invention also may be, but is not limited to, the use of therapeutic agents as adjuvants to opiates to enhance their long-term effectiveness. In addition, or in the alternative, therapeutic agents of the present invention may be beneficial when administered separately or alone to subjects who have previously received or are about to receive an opiate. This may include human suffering from opiate addiction.

The present invention provides a method of managing pain by reducing opiate induced antinociceptive/analgesic tolerance or hyperalgesia by administering a therapeutic agent that reduces ceramide levels in a subject. The inventor has made the surprising discovery that ceramide levels increase after administration of an opiate and that by reducing or preventing this increase, opiate induced antinociceptive/analgesic tolerance or hyperalgesia may be treated or prevented.

Ceramide levels may be reduced by the administration of an agent or agents that inhibit the synthesis of ceramide. Preferable are agents that inhibit the enzymes of the de novo pathway or the production of ceramide from sphingomyelin. Agents that reduce ceramide may be administered prior to opiate treatment, concurrent with an opiate, or subsequent to opiate treatment. Treatment regimens may reduce ceramide levels prophylacticly. A therapeutic agent administered prior to, or concurrent with an opiate may prevent an increase in ceramide caused by administration of that opiate. Ceramide levels may be monitored and treatment regimens modified accordingly to maintain an optimum reduction of ceramide.

Opioid Analgesic Agents

Opiates are well known analgesics, probably best typified by morphine. They operate by mimicking natural peptides such as enkephalins and endorphins to stimulate one or more of the μ- δ- and κ-receptor systems in the nervous system. Opioids are commonly used in the clinical management of severe pain, including chronic severe pain of the kind experienced by cancer patients. (Gilman et al., 1980, Goodman and Gilman's. The Pharmacological Basis of Therapeutics, Chapter 24:494-534, Pub. Pergamon Press; hereby incorporated by reference). The opioids include morphine and morphine-like homologs, including, e.g., the semisynthetic derivatives codeine (methylmorphine) and hydrocodone (dihydrocodeinone) among many other such derivatives. A non-limiting list of opioid analgesic drugs which may be utilized in the present invention include alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, tramadol, salts thereof, complexes thereof, mixtures of any of the foregoing, mixed μ-agonists/antagonists, μ-antagonist combinations salts or complexes thereof, and the like. In certain preferred embodiments, the opioid analgesic is a μ- or κ-opioid agonist. In additional preferred embodiments, the opioid analgesic is a selective κ-agonist.

In certain preferred embodiments, the opioid analgesic is selected from codeine, hydromorphone, hydrocodone, oxycodone, dihydrocodeine, dihydromorphine, diamorphone, morphine, tramadol, oxymorphone salts thereof, or mixtures thereof.

Subjects

Subjects include any mammal, preferable a human mammal. Human subjects include humans who are at risk of developing, or who have developed, opiate induced tolerance or hyperalgesia. This includes any subject who will be or has been administered an opiate. Those particularly at risk of building tolerance are those who require multiple doses of opiates, typically subjects suffering from chronic pain.

Also included are subjects who are addicted to opiates or at risk of addiction to opiates. Subjects who are addicted to opiates may include human subjects who have self administered, and, or, misused opiates, and human subjects who are suffering from hyperalgesia due to opiate withdrawal. Subjects at highest risk for developing opiate induced tolerance or addiction include those who are administered opiates over prolonged periods.

In addition to human subjects are non-human animal subjects such as a primate, a mouse, a pig, a cow, a cat, a goat, a rabbit, a rat, a guinea pig, a hamster, a horse, a sheep, a dog, a cat and the like. Animal subjects include companion animals such as domestic dogs or cats. Also included are trained animals including therapy animals, such as therapy dog. Also included are service animals such as service dogs that assist persons who are handicapped due to loss of sight, loss of hearing, or loss of other facilities. Also included are working animals including dogs or other animals trained for security work. Included are animals maintained for procreation or entertainment purposes including purebred animal breeds or racehorses or workhorses. Included are animals that have been genetically engineered to produce therapeutic proteins, nucleotides or carbohydrates. Also included are rare or exotic animals including zoo animals or wild animals.

Therapeutic Agents

The term “therapeutic agent” refers to any natural or synthesized composition that when administered to a subject relieves the subject of disease or improves health. More specifically, as referred to herein, therapeutic agents include chemical compounds, polypeptides, amino acids, oligonucleotides or combinations thereof, which inhibit ceramide synthesis or reduce ceramide levels in a subject.

Methods of reducing a physiologically substance such as ceramide generally include increasing catabolism, or inhibiting synthesis. The most well known are inhibitors which target the enzymes of ceramide de novo synthesis and the sphingomyelin pathway.

Inhibitors of Ceramide Synthesis

For review, see Delgado et al., (18) hereby incorporated by reference and described below.

De Novo Pathway Inhibitors

The ceramide de novo pathway compromises a series of enzymes leading to ceramide from starting components serine and palmitoyl CoA.

Serine Palmitoyltransferase

Serine palmitoyltransferase (SPT) catalyzes the first step in the synthesis of ceramide, which is the production of 3-ketodihydrosphingosine from serine and palmitoyl CoA. By way of example but not of limitation inhibitors of SPT include the sphingo-fungins, lipoxamycin, myriocin, L-cycloserine and β-chloro-L-alanine, as well as the class of Viridiofungins.

Ceramide Synthase

Ceramide synthase (CerS) catalyzes the acylation of the amino group of sphingosine, sphinganine and other sphingoid bases using acyl CoA esters. By way of example but not of limitation inhibitors of this enzyme include the Fumonisins, the related AAL-toxin, and australifungins. The Fumonisins family of inhibitors are produced by Fusarium verticillioides and includes Fumonisin B1 (FB1). The N-acylated forms of FB1 are known to be potent CerS inhibitors while the O-deacylated form is less potent. Of the N-acylated forms of FB1, the erythro-, threo-2-amino-3-hydroxy-, and stereoisomers of 2-amino-3,5-dihydroxyoctadecanes are also known as CerS inhibitors. Australifungins from the organism Sporomiella australlis is also a potent inhibitor of CerS.

Dihydroceramide Desaturase

Dihydroceramide desaturase (DES) is the last enzyme in the de novo biosynthesis pathway of ceramide synthesis. At least two different forms, DES1 and DES2, are known. By way of example but not of limitation inhibitors of these enzymes include the cyclopropene-containing sphingolipid GT11, as well as a-ketoamide (GT85, GT 98, GT99), urea (GT55) and thiourea (GT77) analogs of this molecule.

Sphingomyelin Pathway Inhibitors

Sphingomyelin hydrolysis by sphingomyelinase (SMases) produces phosphorylcholine and ceramide. At least five isotypes of SMase are known including acid and neutral forms. Several physiological inhibitors of acid SMase have been described including L-α-phosphatidyl-D-myo-inositol-3,5-bisphosphate, a specific acid SMase inhibitor, and L-α-phosphatidyl-D-myo-inositol-3,4,5-triphosphate a non-competitive inhibitor of acid SMase. Ceramide-1-phosphate and sphingosine-1-phosphate have also been described as physiological inhibitors. Glutathione is an inhibitor of neutral SMase at physiological concentrations with a greater than 95% inhibition observed at 5 mM GSH. Compounds, which are structurally unrelated to sphingomyelin, but function as SMase inhibitors included desipramine, imipramine, SR33557, (3-carbazol-9-yl-propyl)-[2-(3,4-dimethoxy-phenyl)-ethyl)-methyl-amine (NB6), Hexanoic acid (2-cyclo-pent-1-enyl-2-hydroxy-1-hydroxy-methyl-ethyl)-amide (NB12) C11AG and GW4869. Compound SR33557 is a specific acid SMase inhibitor (72% inhibition at 30 μM). The compound NB6 has been reported as an inhibitor of the SMase gene transcription. Inhibitors derived from natural sources include Scyphostatin, Macquarimicin A, and Alutenusin, which are non-competitive inhibitors of neutral SMase, and Chlorogentisylquinone, and Manumycin A, which are irreversible specific inhibitors of neutral SMase, as well as α-Mangostin that is an inhibitor of acid SMase. Scyphostatin analogs with inhibitory proprieties include spiroepoxide 1, Scyphostatin and Manumycin A sphingolactones. Sphingomyelin analogs with inhibitory proprieties include 3-O-methylsphingomyelin, and 3-O-ethylsphingomyelin.

The following compounds have been shown to reduce ceramide by inhibition of sphingomyelinase; [3 (10,11-Dihydro-dibenzo[b,f]azepin-5-yl)-N-propyl]-[2 (3,4-dimethoxyphenyl)-ethyl]methylamin, [3 (10,11-Dihydro-dibenzo[b,f]azepin-5-yl)-N-propyl]-[2 (4-methoxyphenyl)-ethyl]methylamin, [2 (3,4-Dimethoxyphenyl)-ethyl]-[3 (2-chlorphenothiazin-10-yl)-N-propyl]-methylamin, [2 (4-Methoxyphenyl)-ethyl]-[3 (2-chlorphenothiazin-10-yl)-N-propyl]-methylamin, [3 (Carbazol-9-yl)-N-propyl]-[2 (3,4-dimethoxyphenyl)-ethyl]methylamin, [3 (Carbazol-9-yl)-N-propyl]-[2 (4-methoxyphenyl)-ethyl]methylamin, [2 (3,4-Dimethoxyphenyl)-ethyl]-[2 (phenothiazin-10-yl)-N-ethyl]-methylamin, [2 (4-Methoxyphenyl)-ethyl]-[2 (phenothiazin-10-yl)-N-ethyl]-methylamin, [(3,4-Dimethoxyphenyl)-acetyl]-[3 (2-chlorphenothiazin-10-yl)-N-propyl]-methylamin, n (1-naphthyl)-N′ [2 (3,4-dimethoxyphenyl)-ethyl]-ethyl diamine, n (1-naphthyl)-N[2 (4-methoxyphenyl)-ethyl]-ethyl diamine, n [2 (3,4-Dimethoxyphenyl)-ethyl]-n [1-naphthylmethyl]amine, n [2 (4-Methoxyphenyl)-ethyl]-n [1-naphthylmethyl]amine, [3 (10.11-Dihydro dibenzo[b, f]azepin-5-yl)-N-propyl]-[(4-methoxyphenyl)-acetyl]-methylamin, [2 (10,11-Dihydro-dibenzo[b, f]azepin-5-yl)-N-ethyl]-[2 (3,4-dimethoxyphenyl)-ethyl]methylamin, [2 (10,11-Dihydro-dibenzo[b,f]azepin-5-yl)-N-ethyl]-[2 (4-methoxyphenyl)-ethyl]-methylamin, [2 (10,11-Dihydro-dibenzo[b,f]azepin-5-yl)-N-ethyl]-[(4-methoxyphenyl)-acetyl]-methylamin, n [2 (Carbazol-9-yl)-N-ethyl]-N′ [2 (4-methoxyphenyl)-ethyl]piperazin, 1[2 (Carbazol-9-yl)-N-ethyl]-4[2 (4-methoxyphenyl)-ethyl]-3,5-dimethylpiperazin, [2 (4-Methoxyphenyl)-ethyl]-[3 (phenoxazin-10-yl)-N-propyl]-methylamin, [3 (5,6,11,12-Tetrahydrodibenzo[b,f]azocin)-N-propyl]-[3 (4-methoxyphenyl)-propyl]methylamin, n (5H-Dibenzo [A, D]cycloheptan-5-yl)-N′ [2 (4-methoxyphenyl)-ethyl]-propylene diamine and [2 (Carbazol-9-yl)-N-ethyl]-[2(4-methoxyphenyl)-ethyl]methylamine, as described in WO2000 EP04738 20000524 herein incorporated by reference.

Also shown to reduce ceramide levels is L-carnitine (200 mcg/ml) as described in U.S. Pat. No. 6,114,385, herein incorporated by reference, as well as silymarin, 1-phenyl-2-decanoylaminon-3-morpholino-1-propanol, 1-phenyl-2-hexadecanoylaminon-3-pyrrolidino-1-propanol, Scyphostatin, L-camitine, glutathione, and human milk bile salt-stimulated lipase as described in U.S. Pat. No. 6,663,850 herein incorporated by reference.

In addition, ceramide levels may be reduced by myriocin, cycloserine, Fumonisin B, PPMP, D609, methylthiodihydroceramide, propanolol, and resveratrol as described in U.S. Patent Application Publication No. 20050182020 herein incorporated by reference. Agents comprised of polypeptides sequences have also been shown to reduce ceramide levels as described in U.S. Pat. No. 7,037,700 and herein incorporated by reference.

This list is non-exhaustive. One of ordinary skill in the art would appreciate that analogs or fragments of the inhibitors included herein would similarly be inhibitory. In addition to the agents described herein are agents that decrease ceramide pathway metabolic enzymes, or increase ceramide catabolic enzymes, including but not limited to agents, which modify, or regulate transcriptional or translational activity or which otherwise degrade, inactivate, or protect theses enzymes.

Therapeutic Reduction of Ceramide Levels

A therapeutic reduction of ceramide may be prophylactic. The inventors have made the discovery that opiate treatment will cause ceramide levels to increase. Therefore, a therapeutic agent that is co-administered, or administered prior to an opiate, may prevent or attenuate an increase in ceramide caused by an opiate. A reduction of this type may be measured against historical data from similar subjects after treatment with opiates. In addition, or in the alternative, therapeutic agents may be administered to reduce baseline levels of ceramide before opiate treatment. Any reduction in ceramide levels which results in attenuation of opiate induced tolerance or hyperalgesia in a subject is a therapeutic reduction. A therapeutic reduction expressed as a decrease in ceramide may be between 0.001% to 10%, 10%-20%, 20%-30%, 30%-40%, 40%-50%, 50%-60%, 70%-80%, 80%-90%, or 90%-100%, preferably greater that 10% of control values. Control values used to calculate a therapeutic reduction include; mean ceramide for similar subjects receiving similar opiate treatment; baseline ceramide determinations made for a particular subject prior to administering a therapeutic agent; or a ceramide determination made for a particular subject after administering an opiate but before treatment with a therapeutic agent. Repeated measurements may be made to continuously monitor ceramide and modify treatment regimens accordingly for optimum therapeutic result.

Determination of Ceramide Levels

A non-limiting method of determining ceramide levels from a subject may be performed as follows: Lipid extracts from blood, plasma, or spinal fluid, may be prepared by back washing with the artificial upper phase and drying under nitrogen prior to storage in chloroform under nitrogen until Electrospray Tonisation Mass Spectrometry (ESI-MS) analyses. Lipid extracts may be mixed with methanol containing 10 mM NaOH prior to direct infusion into the ESI-MS source at a flow rate of 3 μl/min. Ceramides will be directly analyzed in the negative-ion ESI/MS. Tandem mass spectrometry of ceramides after ESI will be performed with collision energy of 32 eV and a collision gas pressure of 2.5 mTorr (argon). With tandem mass spectrometry, ceramides will be detected by the neutral loss of m/z 256.2. Typically, a 5-min period of signal averaging for each spectrum of a ceramide sample, or a 10-min period of signal averaging for each tandem mass spectrum of a lipid extract in the profile mode, will be employed. Ceramide molecular species will be directly quantitated by comparisons of ion peak intensities with that of internal standard (i.e., 17:0 ceramide) in both ESI/MS and ESI/MS/MS analyses after correction for 13C isotope effects.

Ceramide levels may be determined through any number of techniques known to those skilled in the art including but not limited to thin layer chromatography, high-pressure liquid chromatography, mass spectrometry, immunochemical based assays and enzyme based assays, including those using ceramide kinase or diacylglycerol kinase as described by Bektas et al. (Analytical Biochemistry 320 (2003) 259-265), and Modrak (Methods in Molecular Medicine, vol. 111:Vol 2: In Vivo Models, Imaging and Molecular Regulators., Ed. Blumenthal. Humana Press Inc., NJ), and herby incorporated by reference.

Methods of Practicing the Invention

Methods of pharmaceutical administration are well known in the art. They may comprise any form of intravenous, intramuscular, intrathecal, intraperitoneal or subcutaneous injection, ingestion, or absorption suitable for a pharmaceutical composition comprising the therapeutic agent, an opiate analgesic, or both. Administration of a therapeutic agent and opiate may be simulations, or administration of agent may proceed, or be subsequent to opiate treatment by a therapeutic effective period. Non-limiting examples of treatment regimens are described herein.

Treatment Regimen I
1) Administer a therapeutically effective amount of at least one therapeutic agent.
2) Wait a therapeutically effective time.
3) Administer an effective amount of at least one opiate.

Treatment Regimen II
Co-administer a therapeutically effective amount of at least one therapeutic agent and a therapeutically effective amount of at least one opiate.

Treatment Regimen III
1) Administer a therapeutically effective amount of at least one opiate.
2) Wait a therapeutically effective time.
3) Administer a therapeutically effective amount of at least one therapeutic agent.

Treatment Regimen IV
Administer a therapeutically effective amount of at least one therapeutic agent to a subject suffering from opiate induced antinociceptive/analgesic tolerance or opiate induced hyperalgesia.

The herein mentioned treatment regimens may be repeated any number of times, and administration of at least one therapeutic agent or at lest one opiate is not limited to a single application, but may include repeated applications or even continuous infusion.

Therapeutically Effective Amount of Opiate

Opiates are well known and characterized. Non-limiting examples of opiates, their therapeutic effective amounts and equivalent dosages are illustrated in Table 1.

TABLE 1
Opiate Equivalent Dosages (OED)

OED (mg/kg)

| Example | Opiate | IM/IV/SQ | PO | Duration |
|---|---|---|---|---|
| 1 | buprenorphine | 0.4 | — | 4–5 hr |
| 2 | butorphanol | 2 | — | 4–6 hr |
| 3 | codeine | 130 | 200 | 4–6 hr |
| 4 | fentanyl | 0.1 | — | 1–2 hr |
| 5 | hydrocodone | — | 5–10 | 4–5 hr |
| 6 | hydromorphone | 1.3 | 7.5 | 4–5 hr |
| 7 | levophanol | 2 | 4 | 4–7 hr |
| 8 | meperidine | 75 | 300 | 3–5 hr |
| 9 | methadone | 10 | 20 | 4–6 hr |
| 10 | morphine | 10 | 60 | 4–6 hr |
| 11 | nalbuphine. | 10 | — | 4–6 hr |
| 12 | oxycodone | 5–10 | — | 4–6 hr |
| 13 | oxymorphone | 1 | — | 4–6 hr |
| 14 | pentazocine | 30–60 | — | 4–6 hr |

Therapeutically Effective Time

A therapeutically effective time refers to the interval of time between administration of a therapeutic agent and administration of an opiate in treatment regimens where a therapeutic agent is administered prior to an opiate, or subsequent to an opiate. A therapeutically effective time may be determined empirically in each subject by a medical practitioner who may consider among other medically related indicators, a subjects ceramide levels, or ceramide levels from historical data of similar subjects. Non-limiting examples of a therapeutically effective times include; less than 15 minutes; 15 minutes, between 15 minutes and one hour; between 1 and 2 hours; between 2 and 3 hours; between 3 and 4 hours; between 4 and 5 hours; between 5 and 6 hours; between 6 and 7 hours; between 7 and 8 hours; between 8 and 9 hours; between 9 and 10 hours; between 10 and 12 hours; between 12 and 14 hours; between 14 and 16 hours; between 16 and 20 hours; between 20 and 24 hours; between 1 and 2 days; between 2 and 3 days; between 3 and 6 days; more than 6 days.

Therapeutically Effective Amount and Formulation of Therapeutic Agents

Compounds of the invention, including those that are naturally occurring as well as those that are prepared synthetically that inhibit the function or synthesis of one or more enzymes capable of altering ceramide levels, can be administered to a subject at a dosage, effective to provide an inhibition, reduction, or control of ceramide, or opiate induced tolerance or hyperalgesia. Compositions containing therapeutic agents are administered to a patient or subject in an amount sufficient to elicit an effective therapeutic, i.e. opiate tolerance or hyperalgesia reducing, response in the subject. An amount adequate to accomplish this is defined as a “therapeutically effective amount,” a “therapeutically effective dose” or an “effective inhibitory amount.” The dose or amount will be determined by the efficacy or potency of the particular ceramide enzyme inhibitor(s) employed, the opiate employed, dose of opiate, the length of time or frequency of opiate treatment, and the size and condition of the subject including that subject's particular response to opiate treatment. Also for consideration may be ceramide levels in a subject before, during, or after treatment. The size of the dose also will be determined by the existence, nature, and extent of any adverse effects that accompany the administration of a particular compound in a particular subject.

Toxicity and therapeutic efficacy of the substances can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀.

Compounds that exhibit large therapeutic indices are preferred. While compounds that exhibit toxic side effects can be used, care should be taken to design a delivery system that targets such compounds to the site of affected tissue to minimize potential damage to normal cells and thereby reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by lipid extraction of plasma or spinal fluid and determined as described below.

Pharmaceutical compositions for use in the present invention can be formulated by standard techniques using one or more physiologically acceptable carriers or excipients. The compounds and their physiologically acceptable salts, prodrugs, metabolites, or derivatives can be formulated for administration by any suitable route, including via inhalation, topically, sublingually, intranasally, orally, parenterally (e.g., intravenously, intraperitoneally, intramuscularly, subcutaneously, intravesically or intrathecally), or mucosally (including intranasally, orally and rectally). These formulations comprising one or more opiates and therapeutic agents alone or in combination may be supplied in a pre-active form such as a lyophilized power wherein water may be added just before administration to a subject.

For oral or sublingual administration, pharmaceutical compositions of the invention can take the form of, for example, lozenges, tablets or capsules prepared by conventional means with pharmaceutically acceptable excipients, including binding agents, for example, pregelatinized cornstarch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulfate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For intrathecal administration, pharmaceutical compositions of the invention may be delivered in an appropriate vehicle such as saline, by a single injection or as a continuous infusion with the use of a pump such as an osmotic minipump further described below.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Controlled Release Dosage Forms

The therapeutic agent and opioid analgesic combination can be formulated as a controlled or sustained release oral formulation in any suitable tablet, coated tablet or multiparticulate formulation known to those skilled in the art. The sustained release dosage form may optionally include a sustained released carrier which is incorporated into a matrix along with the opioid, or which is applied as a sustained release coating.

The sustained release dosage form may include the opioid analgesic in sustained release form and therapeutic agent in sustained release form or in immediate release form. The therapeutic agent may be incorporated into the sustained release matrix along with the opioid; incorporated into the sustained release coating; incorporated as a separated sustained release layer or immediate release layer; or may be incorporated as a powder, granulation, etc., in a gelatin capsule with the substrates of the present invention. Alternatively, the sustained release dosage form may have the therapeutic agent in sustained release form and the opioid analgesic in sustained release form or immediate release form.

An oral dosage form according to the invention may be provided as, for example, granules, spheroids, beads, pellets (hereinafter collectively referred to as “multiparticulates”) and/or particles. An amount of the multiparticulates which is effective to provide the desired dose of opioid over time may be placed in a capsule or may be incorporated in any other suitable oral solid form.

In one preferred embodiment of the present invention, the sustained release dosage form comprises such particles containing or comprising the active ingredient, wherein the particles have diameter from about 0.1 mm to about 2.5 mm, preferably from about 0.5 mm to about 2 mm.

In certain embodiments, the particles comprise normal release matrixes containing the opioid analgesic with or without the therapeutic agent. These particles are then coated with the sustained release carrier in embodiments where the therapeutic agent is immediately released, the therapeutic agent may be included in separate normal release matrix particles, or may be co-administered in a different immediate release composition which is either enveloped within a gelatin capsule or is administered separately. In other embodiments, the particles comprise inert beads which are coated with the opioid analgesic with or without the therapeutic agent. Thereafter, a coating comprising the sustained release carrier is applied onto the beads as an overcoat.

The particles are preferably film coated with a material that permits release of the opioid (or salt) and if desired, the therapeutic agent, at a sustained rate in an aqueous medium. The film coat is chosen so as to achieve, in combination with the other stated properties, a desired in-vitro release rate. The sustained release coating formulations of the present invention should be capable of producing a strong, continuous film that is smooth and elegant, capable of supporting pigments and other coating additives, non-toxic, inert, and tack-free.

Coatings

The dosage forms of the present invention may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the opioid in desired areas of the gastro-intestinal (GI) tract, e.g., the stomach or small intestine, such that an absorption profile is provided which is capable of providing at least about twelve hour and preferably up to twenty-four hour analgesia to a patient. When a pH-independent coating is desired, the coating is designed to achieve optimal release regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings to obtain formulations may also impart a repeat-action effect whereby unprotected drug is coated over the enteric coat and is released in the stomach, while the remainder, being protected by the enteric coating, is released further down the gastrointestinal tract. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

In certain preferred embodiments, the substrate (e.g., tablet core bead, matrix particle) containing the opioid analgesic (with or without the therapeutic agent) is coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile.

Therapeutically Effective Amounts of Therapeutic Agents

Non limiting examples of therapeutically effective amounts of therapeutic agents may be expressed as a ratio to opiate equivalent dosages (OED, see Table 1) as set out in of Table 2, preferably between an 1:0.1, and 1:100.

TABLE 2
Opiate Equivalent Dosages (OED) and Effective
Amounts of Therapeutic Agents

| | Therapeutic Agent |
|---|---|
| OLD (see Table 1) | (mg) |
| Examples 1–14 | 0.00001 |
| Examples 1–14 | 0.0001 |
| Examples 1–14 | 0.001 |
| Examples 1–14 | 0.01 |
| Examples 1–14 | 0.1 |
| Examples 1–14 | 1 |
| Examples 1–14 | 10 |
| Examples 1–14 | 100 |
| Examples 1–14 | 1000 |
| Examples 1–14 | 10,000 |

The term “pain management” refers to effective use of an analgesic for treating patients suffering from pain, including chronic pain of various etiologies. As used herein it includes the use of opiates and therapeutic agents to enhance their long-term effectiveness while reducing the unwanted side effects that are seen when opiates are administered alone.

Therefore, the invention is drawn to a method of reducing or eliminating opiate induced tolerance or opiate induced hyperalgesia in a subject by administering a therapeutic agent, which reduces ceramide levels by (a) reducing serine palmitoyltransferase activity, (b) reducing ceramide synthase activity, (c) reducing dihydroceramide desaturase, or (d) reducing sphingomyelinase activity.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

It was discovered that repeated administration of morphine increased levels of ceramide in the spinal cord in a murine model. Furthermore, administration of the ceramide synthase inhibitor fumonisin B1 attenuated the development of antinociceptive/analgesic tolerance. Similarly, inhibition of ceramide synthesis by D609, and myriocin, inhibitors of SMase/sphingomyelin synthase and serine palmitoyltransferase respectively, also blocked antinociceptive/analgesic tolerance.

Inhibition of Ceramide Biosynthesis Blocks Morphine Tolerance.

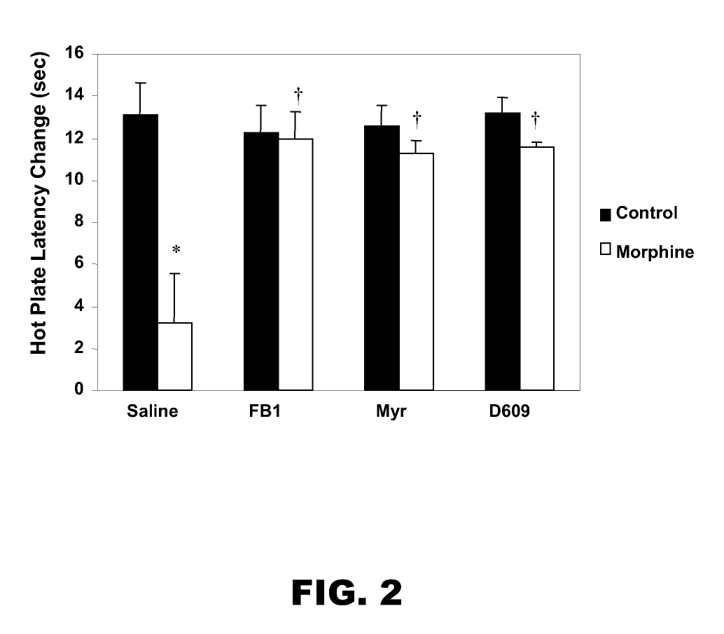

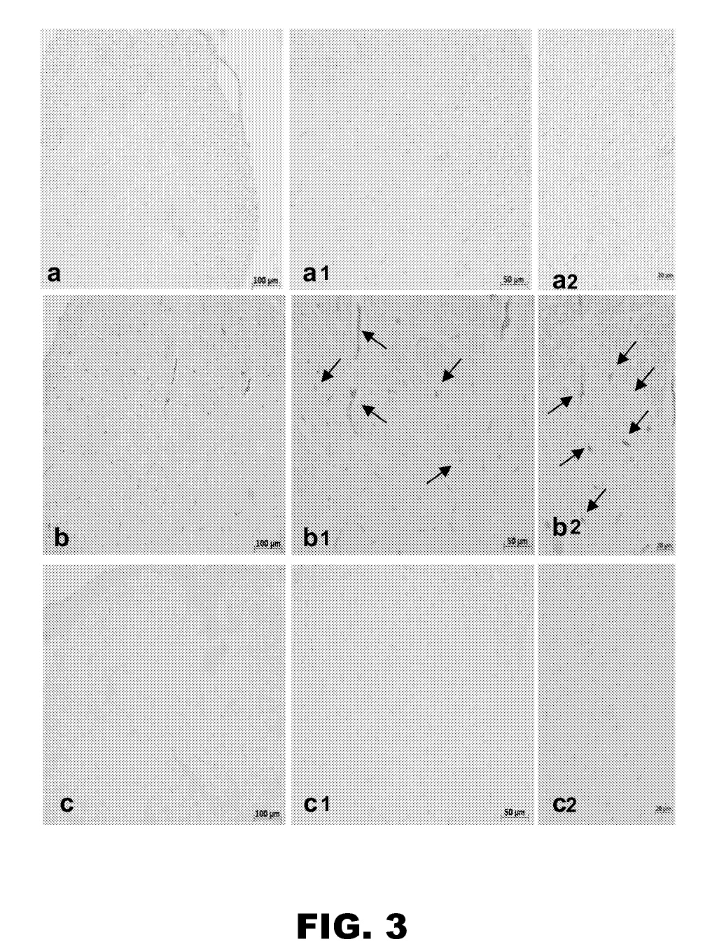

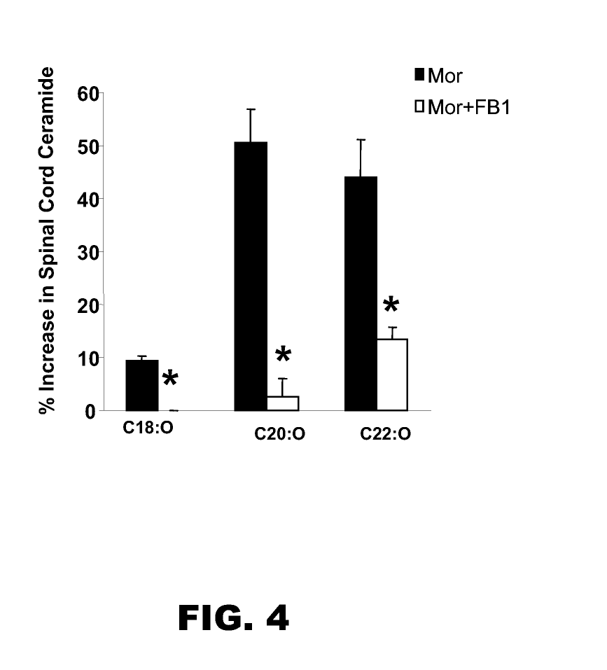

Repeated administration of morphine over 4 days led to the development of antinociceptive tolerance (FIG. 2; from 93±8 to 20±14% MPE for acute morphine in Control vs Morphine groups respectively (P<0.05). This was associated with the appearance of ceramide in the superficial layers of the dorsal horn as detected by immunohistochemistry using an anti-ceramide monoclonal antibody (FIG. 3). As shown by ESI-MS/MS, the predominant ceramide species found to be increased by repeated morphine administration in dorsal horn tissues included 18:0, 20:0 and 22:0 ceramide (FIG. 4; n=3). No staining of ceramide was present in the ventral horn.

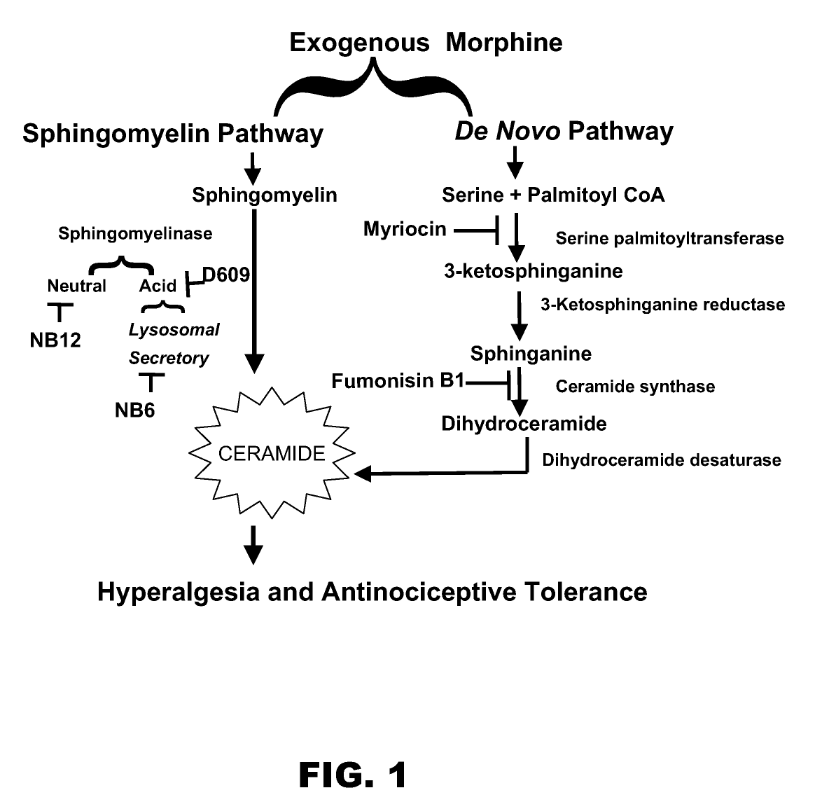

Co-administration of morphine with FB1 (1 mg/kg), prevented the development of antinociceptie tolerance and the increase in ceramide as measured by immunohistochemical analysis and ESI-MS/MS (FIGS. 3, and 4). To address the potential lack of specificity inherent to pharmacological inhibitors such as FB1, we inhibited the upstream enzyme in the de novo pathway, serine palmitoyltransferase, with myriocin (19). Similar to FB1, co-administration of morphine with myriocin (0.2 mg/kg) blocked antinociceptive tolerance (FIG. 2). In order to determine whether activation of the acid spingomyelinase contributed to the development of antinociceptive tolerance, morphine was co-administered with D609 (40 mg/kg). D609, blocked antinociceptive tolerance (FIG. 2). Since D609 has been reported to inhibit ceramide formation also by inhibiting sphingomyelin synthase (the enzyme that generates sphingomyelin, the substrate for SMAse), it is possible that inhibition of both enzymes account for the overall beneficial action of D609 (20, 21). Collectively, these results implicate the participation of the de novo and the sphingomyelin pathways in ceramide biosynthesis (FIG. 1).

The inhibitory effects of these drugs were not attributable to acute antinociceptive interactions with morphine since the responses to acute morphine in the control groups and control groups treated with FB1, myriocin or D609 were similar (FIG. 2). When tested alone these drugs had no antinociceptive effects (not shown).

Induction of Antinociceptive/Analgesic Tolerance in Mice Following Subcutaneous Chronic Delivery of Morphine by Osmotic Minipumps

Antinociceptive/analgesic tolerance was also induced in mice using a continuous infusion of morphine with osmotic minipumps as previously described (22). Thus, the experimental protocol is more clinically relevant than the one using repeated bolus injections. Furthermore, an osmotic pump ensures continuous delivery of morphine without intermittent periods of withdrawal. To this end, we performed pilot testing examining the effects of FB1 in this dosing paradigm. Morphine (50 mg/kg, Morphine groups) or saline (Control groups) was administered to male CD-1 using osmotic minipumps implanted subcutaneously to deliver morphine over 7 days. A total of 4 groups (n=6 mice/group) were used. FB1 (1 mg/kg/day) or an equivalent volume of its vehicle, was given together with morphine by i.p injection once a day for six days. On day six, thirty minutes after the injection of FB1, acute nociception was determined by the tail flick test (Ugo Basile, Italy), with baseline latencies of 4-5 sec and a cutoff time of 10 sec. Latencies were taken in all animals before and 30 minutes after (time point identified from previous studies to produce near-to maximal antinociception) the acute challenge dose of morphine given by intraperitoneal injection (3 mg/kg, i.p) using the tail flick. When compared to the control group, infusion of morphine led to the development of antinociceptive tolerance and this was attenuated in mice that received FB1 (from 90±5% to 15±4% MPE for acute morphine in the control groups and in the Morphine groups respectively, P<0.01 and from 15±4% to 87±4% MPE for acute morphine in the Mor groups and in the Mor+FB1 groups respectively, P<0.01). FB1 did not affect responses to acute morphine (90±5% to 85±6% MPE for acute morphine in the control groups and in the control+FB1 groups respectively).

### General Methods

Induction of Morphine-Induced Antinociceptive Tolerance in Mice.

Nociceptive thresholds were determined by measuring latencies of the mice placed in a transparent glass cylinder on a hot plate (Ugo Basile, Italy) maintained at 52° C. Determination of antinociception was assessed between 7:00 and 10:00AM. Responses indicative of nociception included intermittent lifting and/or licking of the hindpaws or escape behavior. A cut-off latency of 20 sec was employed to prevent tissue damage and results expressed as Hot Plate Latency Changes (response latency-baseline latency, sec). Baseline values ranged between 6-8 sec. Hot plate latencies were taken in mice from all groups on day 5 before (baseline latency) and 40 min after an acute dose of morphine (3 mg/kg, given subcutaneously, sc) (response latency) a time previously identified to produce near-to-maximal anti-nociceptive effect (99±2% antinociceptive effect, n=8). Mice were injected subcutaneously twice a day (at approximately 7AM and 4PM) with morphine (2×10 mg/kg/day; Mor group) or an equivalent volume of saline (0.1 ml, Control group) over four days. Fumonisin B1 (FB1, 1 mg/kg/day), a competitive and reversible inhibitor of ceramide synthase [(19), Cayman Chemical, Ann Arbor, M1), myriocin, an inhibitor of serine palmitosyltransferase (23), D609, an inhibitor of the acid sphingomyelinase (20, 21) or their vehicle (saline, 0.1 ml) were given by daily intraperitoneal (i.p) injection 15 minutes before each morphine dose (Mor+Drug group). On day 5, mice received the first dose of FB1, myriocin, D609 or their respective vehicle followed 15 min later by the acute dose of morphine. In order to exclude a potential interaction between these interventional drugs and acute morphine, mice were treated as in the Control group, except in the presence of the drug under investigation (Control+Drug). On day five, spinal cord tissues from the lumbar enlargement segment of the spinal cord (L4-L6) and dorsal horn tissues were removed and tissues processed for immunohistochemical, Western blot and biochemical analysis as described in the General Methods section. For biochemical determinations of ceramide, the dorsal horn of the spinal cord lumbar segments were harvested and detected by mass spectrometry using electrospray ionization (ESI-MS/MS) and a triple quadrupole mass detector (24). The spinal cord dorsal horn was sampled because the immunohistochemical staining showed that increases in ceramide were presented primarily in this region. Tolerance to the antinociceptive effect of morphine was indicated by a significant (P<0.05) reduction in Hot Plate Latency Change (seq) after challenge with the acute dose. The percent maximal possible antinociceptive effect (% MPE) was calculated as follows: (response latency-baseline latency)/(cut off latency-baseline latency)×100. Six mice per group were used and all experiments were conducted with the experimenters blinded to treatment conditions. Statistical analysis was performed by one-way ANOVA, followed by multiple Student-Newman-Keuls post hoc test.

Light Microscopy.

Spinal cord tissues (L4-L6 area) were taken on day five after morphine treatment. Tissue segments were fixed in 4% (w/v) PBS-buffered paraformaldehyde and 7 μm sections were prepared from paraffin embedded tissues. Tissue trasversal sections were deparaffinized with xylene, stained with Haematoxylin/Eosin (H&E) and studied using light microscopy (Dialux 22 Leitz) in order to study the superficial laminae of the dorsal horn.

Immunohistochemical Localization of Ceramide.

After deparaffinization, endogenous peroxidase was quenched with 0.3% (v/v) hydrogen peroxide in 60% (v/v) methanol for 30 min. Non-specific adsorption was minimized by incubating the section in 2% (v/v) normal goat serum in PBS for 20 min. Endogenous biotin or avidin binding sites were blocked by sequential incubation for 15 min with biotin and avidin (DBA), respectively. Sections were incubated overnight with anti-ceramide antibody (1:50 in PBS, v/v Sigma). Sections were washed with PBS, and incubated with secondary antibody. The counter stain was developed with a biotin-conjugated goat anti-rabbit IgG and avidin-biotin peroxidase complex (DBA brown color) and nuclear fast red (red background). Positive staining are stained in brown. To verify the binding specificity for ceramide, some sections were also incubated with only the primary antibody (no secondary) or with only the secondary antibody (no primary). In these situations, no positive staining was found in the sections indicating that the immunoreactions were positive in all the experiments carried out.

Tissue Preparation and Lipid Analyses by ESI-MS/MS.

Dorsal horn tissues from the lumbar enlargement of spinal cords (50 mg wet weight) were snap frozen and then extracted by the Bligh-Dyer (25) technique in the presence of 1 mg 17:0 ceramide internal standard. Lipid extracts will be back washed with artificial upper phase and then dried under nitrogen prior to storage in 250 ml chloroform under nitrogen until ESI-MS analyses. 50 ml of lumbar spinal cord lipid extract will be mixed with 200 ml of methanol containing 10 mM NaOH prior to direct infusion into the ESI source at a flow rate of 3 ml/min as described by others (24). Ceramides were directly analyzed in the negative-ion mode and detected using tandem mass spectrometry with a collision energy of 32 eV and a collision gas pressure of 2.5 mTorr (argon). With tandem mass spectrometry ceramides will be detected by the neutral loss of m/z 256.2. Typically, a 5-10-min period of signal averaging for each tandem mass spectrum of a lipid extract in the profile mode, were employed. Ceramide molecular species were directly quantitated by comparisons of ion peak intensities with that of internal standard (i.e., 17:0 ceramide) after correction for 13C isotope effects.

### REFERENCES

1. Foley K M. Misconceptions and controversies regarding the use of opioids in cancer pain. Anticancer Drugs 1995; 6 Suppl 3:4-13.
2. Ossipov M H, Lai J, King T, Vanderah T W, Malan T P, Jr., Hruby V J, Porreca F. Antinociceptive and nociceptive actions of opioids. J Neurobiol 2004; 61: 126-148.
3. Ossipov M H, Lai J, Vanderah T W, Porreca F. Induction of pain facilitation by sustained opioid exposure: relationship to opioid antinociceptive tolerance. Life Sci 2003; 73:783-800.
4. Vanderah T W, Ossipov M H, Lai J, Malan T P, Jr., Porreca F. Mechanisms of opioid-induced pain and antinociceptive tolerance: descending facilitation and spinal dynorphin. Pain 2001; 92:5-9.
5. Taylor D A, Fleming W W. Unifying perspectives of the mechanisms underlying the development of tolerance and physical dependence to opioids. J Pharmacol Exp Ther 2001; 297:11-18.
6. Simonnet G, Rivat C. Opioid-induced hyperalgesia: abnormal or normal pain? Neuroreport 2003; 14:1-7.
7. Rothman R B. A review of the role of anti-opioid peptides in morphine tolerance and dependence. Synapse 1992; 12:129-138.
8. Mao J, Price D D, Mayer D J. Mechanisms of hyperalgesia and morphine tolerance: a current view of their possible interactions. Pain 1995; 62:259-274.
9. Celerier E, Rivat C, Jun Y, Laulin J P, Larcher A, Reynier P, Simonnet G. Long-lasting hyperalgesia induced by fentanyl in rats: preventive effect of ketamine. Anesthesiology 2000; 92:465-472.
10. Celerier E, Laulin J P, Corcuff J B, Le Moal M, Simonnet G. Progressive enhancement of delayed hyperalgesia induced by repeated heroin administration: a sensitization process. J Neurosci 2001; 21:4074-4080.
11. Amer S, Rawal N, Gustafsson L L. Clinical experience of long-term treatment with epidural and intrathecal opioids—a nationwide survey. Acta Anaesthesiol Scand 1988; 32:253-259.
12. De Conno F, Caraceni A, Martini C, Spoldi E, Salvetti M, Ventafridda V. Hyperalgesia and myoclonus with intrathecal infusion of high-dose morphine. Pain 1991; 47:337-339.
13. Devulder J. Hyperalgesia induced by high-dose intrathecal sufentanil in neuropathic pain. J Neurosurg Anesthesiol 1997; 9:146-148.
14. Tafesse F G, Temes P, Holthuis J C. The multigenic sphingomyelin synthase family. J Biol Chem 2006; 281:29421-29425.
15. Horinouchi K, Erlich S, Perl D P, Ferlinz K, Bisgaier C L, Sandhoff K, Desnick R J, et al. Acid sphingomyelinase deficient mice: a model of types A and B Niemann-Pick disease. Nat Genet 1995; 10:288-293.
16. Schissel S L, Jiang X, Tweedie-Hardman J, Jeong T, Camejo E H, Najib J, Rapp J H, Secretory sphingomyelinase, a product of the acid sphingomyelinase gene, can hydrolyze atherogenic lipoproteins at neutral pH. Implications for atherosclerotic lesion development. J Biol Chem 1998; 273:2738-2746.
17. Schissel S L, Schuchman E H, Williams K J, Tabas I. Zn2+-stimulated sphingomyelinase is secreted by many cell types and is a product of the acid sphingomyelinase gene. J Biol Chem 1996; 271:18431-18436.
18. Delgado A, Casas J, Llebaria A, Abad J L, Fabrias G, Inhibitors of sphingolipid metabolism enzymes. Biochim Biophys Acta. 2006 December; 1758(12):1957-77.
19. Petrache I, Natarajan V, Zhen L, Medler T R, Richter A T, Cho C, Hubbard W C, et al. Ceramide upregulation causes pulmonary cell apoptosis and emphysema-like disease in mice. Nat Med 2005; 11:491-498.
20. Schutze S, Potthoff K, Machleidt T, Berkovic D, Wiegmann K, Kronke M. TNF activates NF-kappa B by phosphatidylcholine-specific phospholipase C-induced “acidic” sphingomyelin breakdown. Cell 1992; 71:765-776.
21. Luberto C, Hannun Y A. Sphingomyelin synthase, a potential regulator of intracellular levels of ceramide and diacylglycerol during SV40 transformation. Does sphingomyelin synthase account for the putative phosphatidylcholine-specific phospholipase C? J Biol Chem 1998; 273:14550-14559.
22. Vera-Portocarrero L P, Zhang E T, King T, Ossipov M H, Vanderah T W, Lai J, Porreca F. Spinal NK-1 receptor expressing neurons mediate opioid-induced hyperalgesia and antinociceptive tolerance via activation of descending pathways. Pain 2006.
23. Erdreich-Epstein A, Tran L B, Cox O T, Huang E Y, Laug W E, Shimada H, Millard M. Endothelial apoptosis induced by inhibition of integrins alphavbeta3 and alphavbeta5 involves ceramide metabolic pathways. Blood 2005; 105:4353-4361.
24. Han X, Gross R W. Shotgun lipidomics: electrospray ionization mass spectrometric analysis and quantitation of cellular lipidomes directly from crude extracts of biological samples. Mass Spectrom Rev 2005; 24:367-412.
25. Bligh E G, Dyer W J. A rapid method of total lipid extraction and purification. Can J Biochem Physiol 1959; 37:911-917.

1. A method of reducing the development of opiate induced antinociceptive tolerance associated with an administration of an amount of opiate to a subject, the method comprising, administering to the subject the amount of opiate, and concurrently administering a therapeutically effective amount of one or more agents that inhibit ceramide synthesis, whereby the development of the opiate induced antinociceptive tolerance associated with the amount of opiate is reduced.

2-3. (canceled)

4. The method of claim 1, wherein the method of administering is selected from the group consisting of intravenous, intramuscular, intrathecal, intraperitoneal, subcutaneous injection, or ingestion.

5-6. (canceled)

7. The method of claim 1, wherein one or more agents that inhibit ceramide synthesis consist of agents which inhibit serine palmitoyltransferase.

8. The method of claim 7, wherein the agent which inhibits serine palmitoyltransferase is myriocin.

9-16. (canceled)

17. The method of claim, 1 wherein the subject is a human subject and opiate is selected from the group consisting of morphine, diamorphine, hydromorphone, oxymorphone, pethidine, levophanol, methadone, meperidine, fentanyl, codeine, hydrocodone, oxycodone, propoxyphene, buprenorphine, butorphanol, pentazocine and nalbuphine.

18. The method of claim 1, whereby the opiate equivalent dosage to agent ratio is from 1:0.2 to 1:40.

19-33. (canceled)

34. The method of claim 1, further comprising a course of treatment:
a. whereby the course of treatment consists of the method of claim 1 repeated at least once,
b. whereby the development of the opiate induced antinociceptive tolerance associated with the course of treatment is reduced.

35. The method of claim 1, whereby the administration of opiate is by systemic administration.

36. The method of claim 1 whereby the administration of ceramide synthesis inhibitor is by systemic administration.

37. A method of reducing the development of opiate induced antinociceptive tolerance associated with an amount of opiate and a course of treatment, the method comprising:
a. administering the amount of opiate over the course of treatment, the course of treatment comprising one or more administrations of opiate;
b. whereby each administration of opiate is concurrent with a therapeutically effective amount of one or more ceramide synthesis inhibitor;
c. thereby reducing the development of opiate induced antinociceptive tolerance associated with the amount of opiate.

38. The method of claim 37, whereby the administration of opiate is by systemic administration.

39. The method of claim 37, whereby the administration of ceramide synthesis inhibitor is by systemic administration.

40. The method of claim 37, wherein the method of administering is selected from the group consisting of intravenous, intramuscular, intrathecal, intraperitoneal, subcutaneous injection, or ingestion.

41. The method of claim 37, wherein one or more agents that inhibit ceramide synthesis consist of agents, which inhibit serine palmitoyltransferase.

42. The method of claim 41, wherein the agent, which inhibits serine palmitoyltransferase, is myriocin.

43. A method of reducing the development of opiate induced antinociceptive tolerance in a subject, associated with an amount of opiate, the method comprising:
a. administering an amount of opiate through repeated or continuous administration;
b. whereby the opiate is administered concurrently or within a therapeutically effective time of a therapeutically effective amount of one or more agents which inhibit ceramide synthesis;
c. whereby the administration of both the opiate and the ceramide synthesis inhibitor is by systemic administration.

44. The method of claim 43, wherein the method of administration is selected from the group consisting of intravenous, intramuscular, intraperitoneal, or ingestion.
